(19) United States
(12) Patent Application Publication
(10) Pub. No.: US 2023/0213225 A1
(43) Pub. Date: Jul. 6, 2023

(54) AN ISOLATION CHAMBER

(71) Applicants: THE BIOFACTORY PTE LTD, Singapore (SG); SINGAPORE HEALTH SERVICES PTE LTD, Singapore (SG)

(72) Inventors: Hairil Rizal ABDULLAH, Singapore (SG); Mavis TEO, Singapore (SG); Antonia ZENG, Singapore (SG); Zihui TAN, Singapore (SG); Gabriel TAN, Singapore (SG); Chun Siong LEE, Singapore (SG); Ron WIGHT, Singapore (SG); Marcus Hiong Tat TAN, Singapore (SG)

(73) Assignees: THE BIOFACTORY PTE LTD, Singapore (SG); SINGAPORE HEALTH SERVICES PTE LTD, Singapore (SG)

(21) Appl. No.: 18/000,478

(22) PCT Filed: Aug. 31, 2020
(86) PCT No.: PCT/SG2020/050505

§ 371 Date: Dec. 1, 2022

(30) Foreign Application Priority Data
Jun. 1, 2020 (SG) 10202005153X

(51) Int. Cl. F24F 7/06; A61G 10/00; A61L 2/20; A61L 2/26; E04H 1/12; E04H 3/08

(52) U.S. Cl. CPC F24F 7/06; A61G 10/005; A61L 2/208; A61L 2/26; E04H 1/125; E04H 3/08; A61L 2202/25; F24F 2011/0002

(57) ABSTRACT

An isolation chamber includes a plurality of walls and ceiling defining an internal chamber and a first and second door. The doors seal engagement with the walls. A fan filter unit is arranged to extract air from the internal chamber. The isolation chamber is arranged to couple to a doorway, and the coupling arranged to provide an airtight seal about the doorway.

### FIELD OF THE INVENTION

The invention relates to the control of airborne virus within a healthcare facility. In particular, the invention relates to systems and methods for preventing such viral spread through control and diversion of air flow.

### BACKGROUND

Traditionally, hospitals have constructed negative pressure rooms for isolating patients with infectious disease. The key principle is that when an aerosol generating procedure is performed within these negatively pressured rooms, air will flow from the relatively higher pressure corridor into these rooms until their relative pressures have been equalized. These infective aerosols will not exit the room affect the corridors and the other patients in the corridor.

There are existing standards which define the pressure differential between the room and corridor i.e. more than 2 Pa to ensure that this one-way air flow is achieved.

To construct one of these rooms, there needs to be a steady state imbalance between air supplied into the room and air removed from the room. To create a negatively pressured room, the idea is to remove air faster than it is supplied. The number and construction of ducts, the exhausting capacity via venturi valves must all be designed and computed beforehand.

These rooms are very complex and expensive to construct and maintain. More importantly, it will require considerable construction work to convert a normal room into a negatively pressured one.

The idea of a negatively pressured room cannot be applied to an operating theatre as there is no feasible solution for the following reasons:
Difficulty in ensuring air tightness of large operating theatre hence it is difficult to ensure negative pressure consistently
Difficulty in ensuring air tightness of large operating theatre means that by virtue of negative pressure, contaminants may inadvertently come from different parts of hospital

For already constructed pressure neutral rooms, with either environmental conditioning (cooling or heating), if there are patients with infectious disease in those rooms, healthcare professionals are required to treat those patients in personal protective clothing or equipment (‘PPE’).

Donning and doffing refers to the practice of putting on and taking off, personal protective clothing (PPE). For healthcare related applications, donning is performed within an environmentally controlled space prior to entering an area exposed to chemical, biological and radiological (CBR) agents.

Doffing is performed immediately after exiting the exposed area. Donning and doffing requires a controlled space that is a safe and effective intermediate barrier that contains and prevents the transmission of CBR agents. Such intermediate barriers are integral to the planning and construction of the facility that requires the person(s) to wear PPE. Air showers or airlock chambers are an essential feature of these barriers. The person who has donned the PPE will pass through the air shower prior to entering the exposed area and upon exiting will again pass through the air shower. Provision for safe disposal of PPE gear, hand washing and provision of hand sanitization is essential. In addition to the forced removal and filtering of air afforded by the air shower, germicidal radiation is increasingly being employed within these intermediate barriers as a means to reduce biological hazards during doffing and to protect sanitary workers who will then dispose of the PPE and or clean the exposed surfaces of the intermediate barrier.

With the incidence of large-scale infectious disease and pandemic becoming increasingly prevalent, healthcare facilities such as hospitals must segregate highly infectious patients. In most instances these hospitals were not constructed with controlled spaces and intermediate barriers to contain highly communicable diseases.

### SUMMARY OF INVENTION

In a first aspect, the invention provides an isolation chamber comprising: a plurality of walls and ceiling defining an internal chamber; a first and second door, the doors sealing engagement with the walls; a fan filter unit arranged to extract air from the internal chamber; said isolation chamber is arranged to couple to a doorway, said coupling arranged to provide an airtight seal about said doorway.

Therefore, by providing an isolation chamber having a fan filter unit, air from the external environment and the isolated room to which the isolation chamber is mounted remains discrete from each other preventing airborne particles from escaping, or entering, the room intended for isolation.

The isolation chamber may be a portable and mobile system, and in some embodiments be considered an anteroom. The system may comprise fans to blow air into and air out of the isolation chamber and one-way direction flaps to ensure that air flows from a clean space into the patient room.

In a further embodiment, positive pressure or negative pressure differential may be provided by the isolation chamber.

‘Positive pressure’ System—In this instance the fan filter unit may draw in air from a clean space, filter and disinfect the air and pump it into the isolation chamber. The doors are hermetically sealed and thus air can only escape via outlets that are fitted with one way flaps. These one way flaps may be in arranged such that air is being exhausted to the source of the CBR agents (i.e. patient room)

‘Negative pressure’ System—In this instance the fan filter unit may pull air out of the isolation chamber, filter and disinfect the air and then pump the air into the room containing a CBR source. Again the doors are hermetically sealed and thus the air can only enter the isolation chamber via inlets which are fitted with one way flaps (similar to one way flow valves) and these one way flaps are arranged in such a way that air is pulled in from the clean space outside the isolation chamber. The one-way flaps may be designed in a fashion to be counteracted by a change in pressure differential. For example, the one-way flap may be designed to open when the isolation chamber is of lower pressure than the corridor. When the room-side doors of the isolation chamber opens to a higher pressure patient room, ‘contaminated’ air from the patient room will flow into the isolation chamber. However, due to the equalization and subsequent reversal of pressure differential between the isolation chamber and corridor, the one-way flap will shut off, preventing room air from leaking into the corridor through the one-way flap.

In one embodiment, the isolation chamber may be wheeled into a modest sized lift and moved around hallways within a hospital and wheeled into position against an existing structural door frame. It should be noted that, in this embodiment, instead of drawing or exhausting air from existing hospital infrastructure, it may be drawing and exhausting air into or out of the rooms.

In one embodiment, the isolation chamber may be constructed from two concertinaed rectangular arches or frames that are mounted on lockable castor wheels. One section with a double door opening that is sized to nest within a larger rectangular section with double door.

Both frames may be locked together when in transit and rendered mobile by the castors. The castors may selectively project from the bottom of the unit to grant mobility to the unit and be retracted into the unit to allow for the unit be firmly interfacing the floor.

### BRIEF DESCRIPTION OF DRAWINGS

It will be convenient to further describe the present invention with respect to the accompanying drawings that illustrate possible arrangements of the invention. Other arrangements of the invention are possible and consequently, the particularity of the accompanying drawings is not to be understood as superseding the generality of the preceding description of the invention.

FIGS. 1A to 1H are various views of an isolation chamber according to one embodiment of the present invention;

FIGS. 2A to 2L are sequential views of the operation of an isolation chamber according to a further embodiment of the present invention;

FIGS. 3A and 3B are a computational fluid dynamics model for an isolation chamber according to a further embodiment of the present invention;

FIGS. 4A and 4B are isometric views of an isolation chamber according to a still further embodiment of the present invention;

FIGS. 5A to 51 are sequential views of the operation of an isolation chamber according to a still further embodiment of the present invention;

FIGS. 6A and 6B are a schematic and an isometric view of an air flow pattern according to one embodiment of the present invention;

FIGS. 7A and 7B are a schematic and an isometric view of an air flow pattern according to a further embodiment of the present invention;

FIGS. 8A and 8B are a schematic and an isometric view of an air flow pattern according to a further embodiment of the present invention;

FIGS. 9A and 9B are a schematic and an isometric view of an air flow pattern according to a further embodiment of the present invention;

FIGS. 10A and 10B are a schematic and an isometric view of an air flow pattern according to a further embodiment of the present invention;

FIGS. 11A and 11B are a schematic and an isometric view of an air flow pattern according to a further embodiment of the present invention;

FIGS. 12A and 12B are isometric views of an isolation chamber according to a further embodiment of the present invention;

FIGS. 13A and 13B are isometric views of an isolation chamber according to a further embodiment of the present invention;

FIGS. 14A and 14B are isometric views of an isolation chamber according to a further embodiment of the present invention;

FIGS. 15A and 15B are isometric views of an isolation chamber according to a further embodiment of the present invention;

FIGS. 16A to 16C are isometric views of an isolation chamber according to a further embodiment of the present invention;

FIG. 17 is an isometric view of an isolation chamber according to a further embodiment of the present invention, and;

FIGS. 18A to 18C are decontamination characteristics -v- time.

### DETAILED DESCRIPTION

At present, most emergency departments, ICU and operating theatres do not have a dedicated airborne infection isolation room. Most of these areas are positive pressured with filtered air (such as an operating theatre) to the surrounding area, including common corridors, to act as a protective environment for patients. However, when the patient is the source of the contamination, this dispersive cascade of positive pressure driven flow increases the risk of contamination for everyone else in the surrounding area.

Accordingly, the present invention provides an isolation chamber which is arranged to receive and divert airflow according to the requirements of the room to which it is directed.

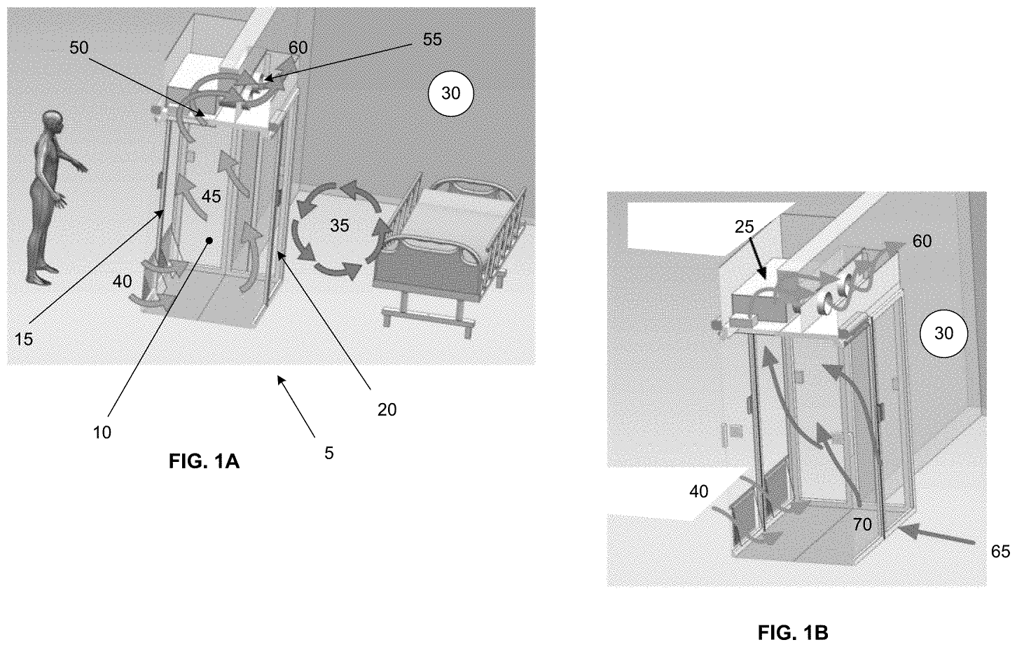

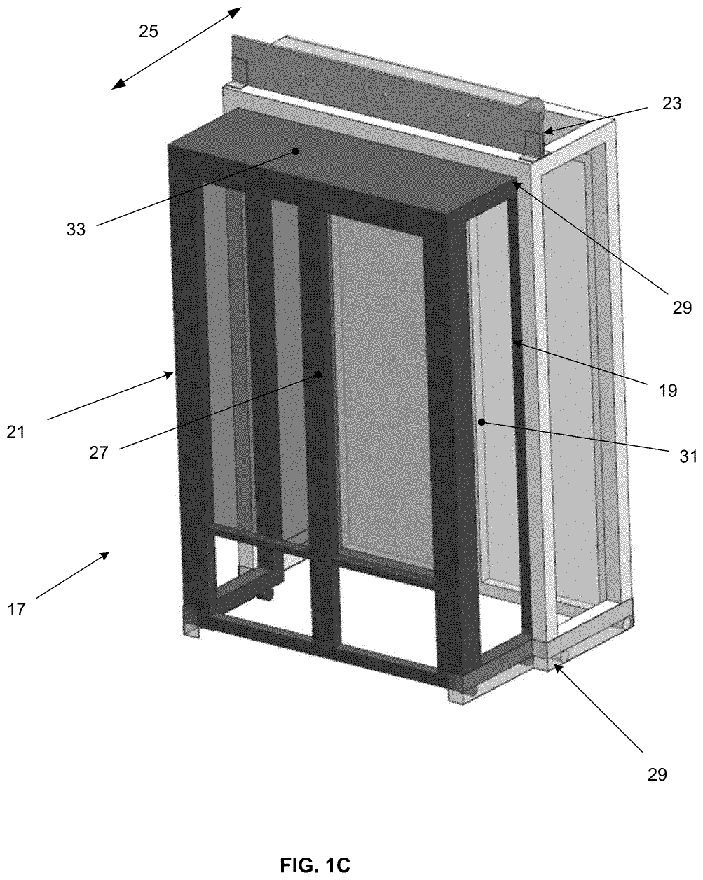

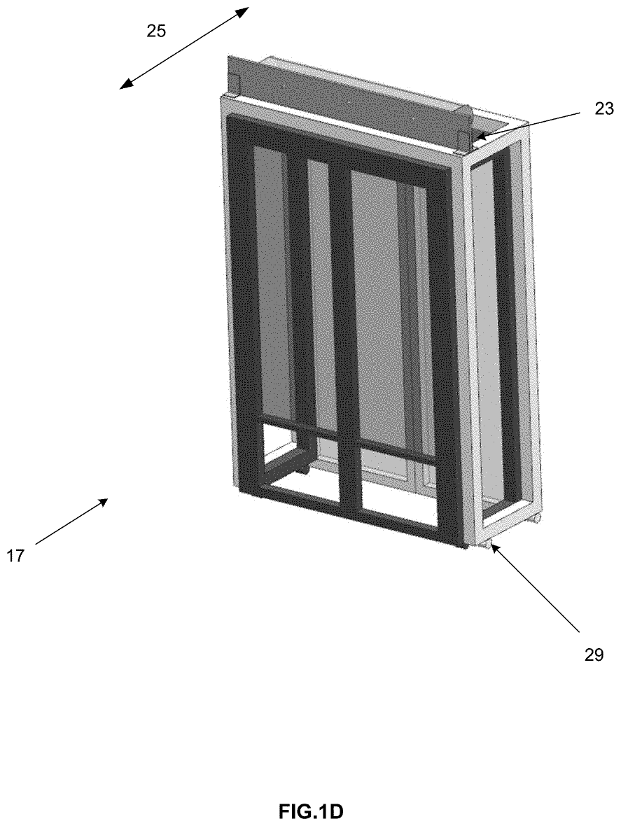

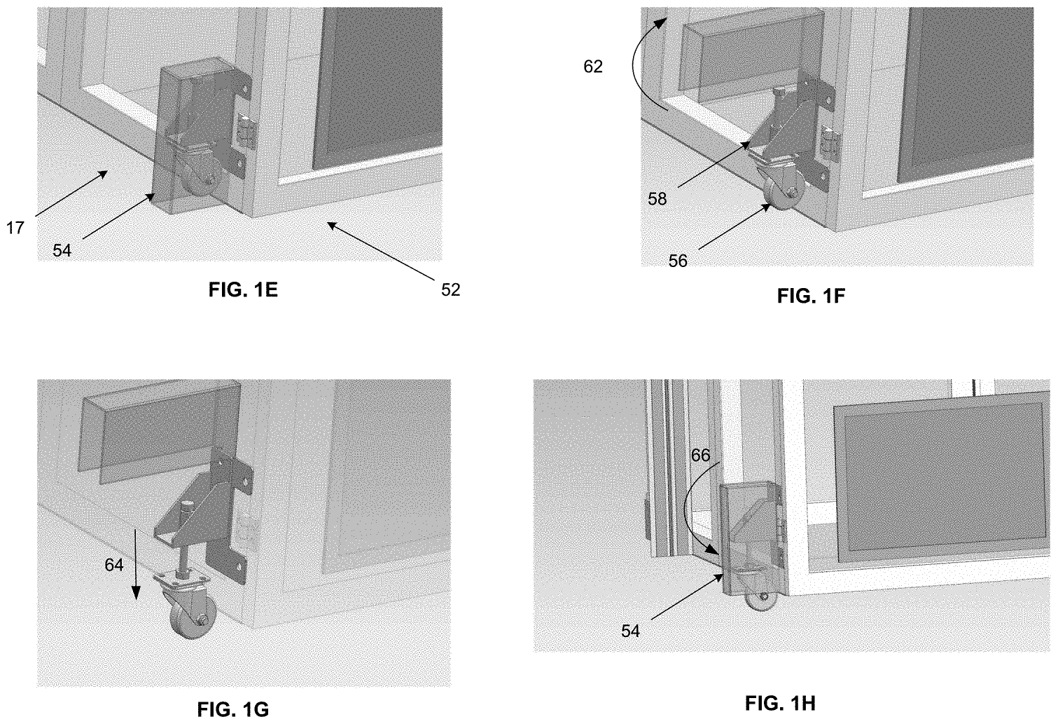

In one embodiment, FIGS. 1A to 1H show various embodiments of an isolation chamber according to the present invention. FIGS. 1A and 1B show an isolation chamber 5 comprises an external door 15 and an internal door 20 with walls and the ceiling which define an internal chamber 10. The isolation chamber 5 is arranged to be mounted to a doorway of a room 30 so as to ensure that air within the room 30 circulates 35 without contaminating the external environment. The isolation chamber 5 is arranged to allow a person to enter the room whilst maintaining the potentially contaminated air to be contained and thus converting the room 30 into an isolation room.

When the isolation chamber 5 is mounted to the door opening, one door will be on the outer side (which variously may be described as the dirty or corridor side) and one on the isolation room side. To facilitate opening for medical staff not wishing to contaminate themselves, the doors of the isolation chamber may have an opening switch that may be operated by an elbow. Alternatively, opening of the doors may be by a proximity sensor, and so a hand movement may open the door. As will be described later, during decontamination of the isolation chamber or room, such switches (and other means of opening the door) may be disabled until the process has been completed. The door may then open automatically, or may require the entrant to activate the switch.

The isolation chamber 5 further comprises a fan filter unit 25, which may include a H13 HEPA filter which filters up to 99.95% of 0.1 micron particles per litre of air. The fan filter unit with HEPA filtration may be arranged to provide sufficient velocity (for instance, 350-400 air changes per hour) to maintain negative pressure and unidirectional airflow into the isolation room 30 during operation.

It will be appreciated that other filter systems may be used, including chemical filters, activated charcoal and regenerative filters.

The fan filter unit is arranged, such that air 40 entering the chamber from the external environment is directed 45 into an outlet 50 so that the fan filter unit 25 can vent 60 the air back into the isolation room 30. Similarly, air entering 65 the isolation chamber 10 from the isolation room is directed to the outlet 50 by the fan filter unit 25 to be vented back into the isolation room 30. Thus, the isolation chamber 5, by providing sealed doors 15 and 20 and a fan filter unit, prevents the cross-circulation of contaminated air from the isolation room 30 to the external environment.

The isolation chamber 5 may be arranged such that mechanically, through magnetic lock or other sch arrangement, the internal and external doors are prevented from being opened at the same time. Such an arrangement may also have a time lock, such that a designated period of time must pass before the second door can be opened following closure of the first. Such a time period may be to ensure the fan filter unit can extract all the air from the internal chamber, ensuring no contaminated air escapes. Such a time period may be reduced, or unnecessary, when entering from the external environment into the isolation room, if the contaminants reside in the isolation room. The alternative may be to ensure no contaminated air enters the isolation room (if used for high risk patients).

The isolation chamber further includes a control system to monitor and control activity related to the use of the isolation chamber. For instance, the control system may control the pressure within the isolation chamber according to certain criteria. Such criteria may include running at high flow rate for high air exchange and differential pressure to purge contaminated air (“decontamination cycle”). Alternatively, it may run at a low flow rate to maintain required differential pressure

Further, the isolation chamber may include sensors to detect entry/exit of personnel from either dirty or clean side, with the control system running an appropriate decontamination cycle by locking the interlocked doors for certain calibrated time periods.

The control system may be able to synchronise speed or selectively operate fan filter unit (proximal) closest to patient room to create more localised ‘sinks’ which act to pull in contaminated air before it reaches the distal end of the isolating chamber.

Further, the control system may be able to employ pressure control feedback during continuous low speed to minimise impact on patient room air handling unit and lower energy consumption.

Further still, the control system may be able to trigger, or detect through sensors, that a patient room is being fumigated (intra patient decontamination) and enter a decontamination mode in which the patient room facing doors are kept open, fan filter units run at constant low speed and outer doors are locked. This will ensure entire air handling subsystems are decontaminated and continues to be effective at providing a protected environment

In a further embodiment, shown in FIG. 1C, the isolation chamber 17 may be selectively movable such that it may be moved from one location to another so as to best use the available facilities within the hospital. FIGS. 1C and 1D show that the isolation chamber 17 may be collapsible 25 for easier transport, then expanded 25 telescopically. A nested section 21 having a door 27 mounted thereto, may have slides 29 for expanding 25 from the main section 19, in which is mounted another door. In this embodiment, the isolation chamber 17 may seal into a doorway using a seal 23, with the nested section 21 sliding into the room. It will be appreciated that the nested section may have the door frame seal instead. Similarly, the fan filter unit may be movable from an expanded operational position to a retracted position within the isolation chamber ceiling to facilitate movement, such as in an elevator.

Depending on whether it is configured to be a ‘positive pressure’ system or a ‘negative pressure system’, the fan filter unit (‘FFU’) may correspondingly be configured to either blow or draw air from the isolation chamber. Filters may be mounted before or after the fan. The FFU may be mounted on the top of one of the frames. In a ‘positive pressure’ system, the FFU may be blowing air into the isolation chamber, and it may be mounted on the section on the ‘clean’ side. In a ‘negative pressure’ system, the FFU may draw air from the isolation chamber and it may be mounted on the section on the ‘dirty’ side where the CBR source is located.

The FFU may include UVC light bulbs that are positioned before or after the fan or filter. The UVC radiation within a wavelength of 240-280 nm can disrupt the chemical structure of RNA and DNA preventing micro-organisms from reproducing. This UVC light may disinfect the fan and HEPA filter of the fan filter unit. The UVC lights may be recessed or hidden from direct vision by forming appropriate ducts around the lamps. This may allow the UVC lights to continuously decontaminate the FFU while preventing accidental UV light exposure to people during use.

When wheeled into position, the double doors of the smaller section may face the patient ward whilst the outer section may be positioned against the structural door section of the patient ward. The smaller section is unlocked and wheeled out into the general ward and locked into position. This may create an occupied space sealed by interlocked double doors, creating an intermediate barrier between the general ward and the hallway. Or vice versa.

The fan filter unit may be mounted in such a way as to pull air out and the one-way flaps may allow air to flow into the isolation chamber (left in the figure below). The fan filter unit may be mounted in such a way as to push air in and the one-way flaps may allow air to flow out of the isolation chamber.

To fit through door openings, a deployable air handling unit may be raised from a lowered position. This feature may only be used when the 2 rectangles of the system are fully extended. In the raised position, the FFU forms a tight seal to the top of the section.

Additionally, self-closing flaps may be mounted before and after the FFU to allow for one-way direction of air flow. This allows for preservation of the air pressure in the isolation chamber when the FFU stops operation and acts as a protective shield preventing airborne particulates from being deposited onto the FFU. The flaps may be passive or actively controlled. When the flaps are passively controlled, they may operate as a cantilever with one end of the lever being the covering flap and the other end being a calibrated weight. When the weight and lever lengths are appropriately selected, this allows the flap to be easily moved by air flow, against the covering flap. This allows air to flow freely past the flaps. When the FFU stops, the flap will tend to drop down, closing the lumen.

Similarly, self-closing flaps may also be installed on the sections to allow for one-way direction of air flow. These flaps are typically installed on the opposite frame without the FFU, to allow for better air exchange within the isolation chamber, and act in unison with the FFU operation. These flaps may be active or passively controlled. For example, in a ‘negative pressure’ system, the FFU draws air from the isolation chamber and expels it into the room with CBR. The flap is mounted on the ‘clean’ side and allows air from the clean space into the isolation chamber. When the flaps are passively controlled, they may operate as a lever with a single fulcrum point, and one end of the lever being the covering flap and the other end being a calibrated weight. The weight or lever arm may be calibrated such that the flap will selectively open, allowing for air to flow past the flaps when the FFU is in operation and sufficient pressure differential between either side of the flap, has been reached. The size of the flaps may also be configured be relative to the size of the isolation chamber and the intended air exchange rate within the isolation chamber.

To achieve effective pressure management of the isolation chamber when the doors are closed, the air flow is only selectively enabled to and from the isolation chamber via the flaps and the FFU. All other seams or gaps are appropriately sealed off. The seam between the doors and frames are lined with appropriately selected gaskets that form a slight interference fit and cover the seams between the door and frame when the doors are closed. Additional surmountable gaskets may be oriented on both sides of the door frame such that the door is able to passively resist against external forces and maintain a tight seal to the door frame when faced with a sudden surge in air pressure (for example when the door on the opposite frame is opened and the opposite room is vastly higher or lower pressure than the isolation chamber).

To assist with the re-location of the isolation chamber, the chamber may include a pair of wheel assemblies, with one of said assembly 52 shown in FIGS. 1E to 1H. The assembly includes a cover 54 which is arranged to flip 62 up (or may slidingly retract) exposing the bracket 58 and wheel m56. The wheel is then arranged to extend 64 from the bracket until it is in a position to contact the ground clear from the isolation chamber. In this position, the isolation chamber can longer sit flush on the ground, but is slightly elevated by the pair of wheels projecting from the bracket 58. The protective cover then flips 66 back ready to tilt the isolation chamber and move to a new location. It will be appreciated that four or more wheel assemblies may be positioned about the base of the isolation chamber. Once projecting down, the four wheels can then lift the chamber clear and allow the chamber to be moved to a new location similar to a trolley.

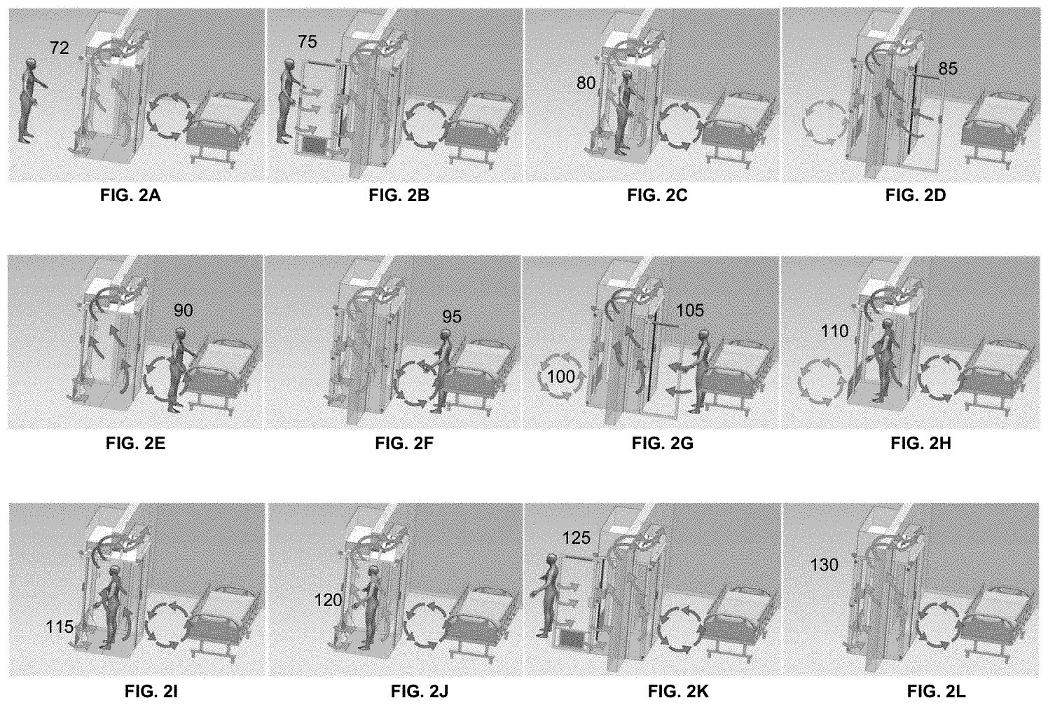

FIGS. 2A to 2L show the sequential steps of the person entering 75 the isolation chamber whereby air from the external corridor is directed 80 out of the isolation chamber into the isolation room. Wirth the internal and external doors sealed, FIG. 2C shows the flow of air as well as the undisturbed circulation of air within the isolation room. On opening the internal door 85 air from the isolation room is directed through the outlet and redirected back into the room until the isolation chamber is sealed by closing the internal door. Thereafter, the person 95 resides within the isolation room. On exiting the internal door opens 100 and as indicated in FIG. 2D internal airflow is redirected by the fan filter unit back into the isolation room without disturbing the circulation of air 100 within the corridor. On sealing the internal and external door, air within the internal chamber 10 is extracted through the fan filter system and so allowing for the external door to open 115 and the person to exit 120, 125. Finally, on closing the external door 130 the internal chamber 10 has air extracted once again and thus allowing the external door to open without potentially contaminated air within the isolation room from escaping.

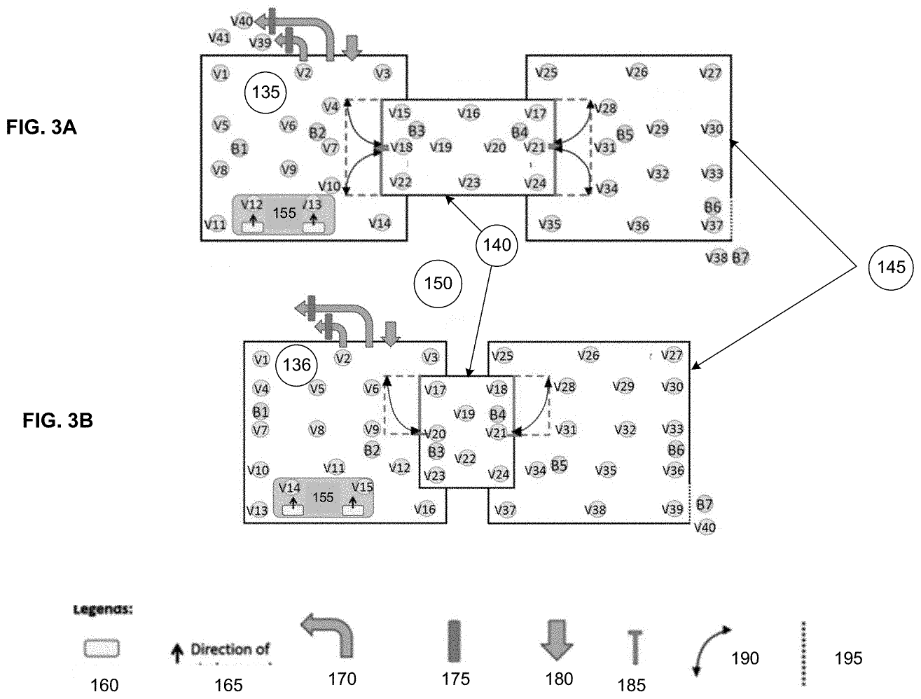

FIGS. 3A and 3B show models used to test the efficacy of the isolation chamber according to the present invention. Located in the model are nebulizers 160 positioned on a patient's bed 155 which project 165 the viral aerosol outwards into the room 135 in which the patient is located. For FIG. 3A, the test is conducted in an operating theatre and for FIG. 3B in an ICU room. HEPA scrubbers 175 are placed each of the room vents 170, with measurements taken outside 150, with inlets 180 unfiltered. An isolation chamber 140 according to the present invention is placed intermediate the room 135 in which the patient is located and the corridor 145.

| | | Estimated | | Estimated | | Estimated |
|---|---|---|---|---|---|---|
| | Estimated | Total PFU | Estimated | total PFU | Estimated | total PFU |
| | Average | based upon | Average | based upon | Average | based upon |
| | PFU per | sum of V1 | PFU per | sum of V15 | PFU per | sum of V25 |
| | plate | to V1 | plate | to V24 | plate | to V37 |
| Operating | 1,551 | 21,713 | 99 | 993 | 0 | 0 |
| Theatre | | | | | | |
| FIG. 3A | | | | | | |
| | | Estimated | | Estimated | | Estimated |
| | Estimate | Total PFU | Estimated | total PFU | Estimated | total PFU |
| | Average | based upon | Average | based upon | Average | based upon |
| | PFU per | sum of V1 | PFU per | sum of V17 | PFU per | sum of V25 |
| | plate | to V16 | plate | to V24 | plate | to V37 |
| ICU | 10,290 | 164,642 | 267 | 2,140 | 0 | 0 |
| FIG. 3B | | | | | | |

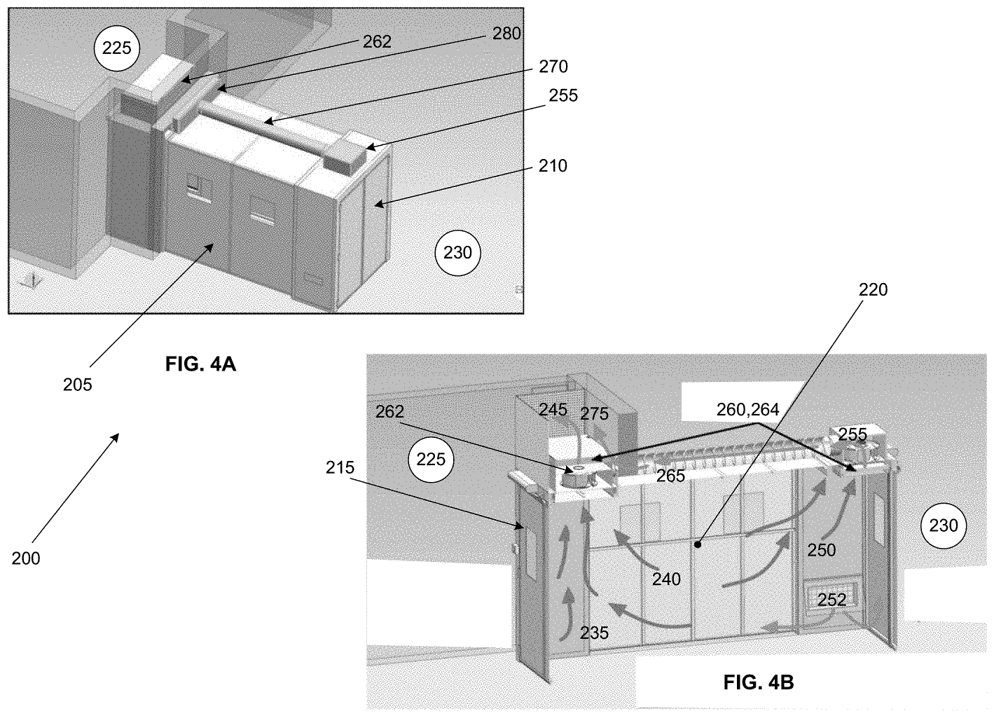

FIGS. 4A and 4B show an alternative embodiment of the isolation chamber 200 having an elongate section 205 with external door 210 and internal door 215. Where the embodiment differs from that of FIGS. 1A to 1C is having the internal elongate chamber 220 within the isolation chamber 200. Because of the size of the internal chamber 220, a larger volume of air needs to be extracted when the doors are sealed and so two fan filter units 255, 262 are provided. As before the fan filter unit 262 resides close to or, in this case, within the isolation room 225. The fan filter unit 262 is arranged to accommodate the extraction of air 235, 240, proximate to the internal door 215. However, to ensure that air closer to the external door 210 leading to the external environment 230 is also extracted air 250 is removed through fan filter unit 255.

The fan filter unit 255 is connected by ducting 270 to an inlet 280 so as to provide fluid communication between the duct 270 and the fan filter unit 262. Thus, air 275 from the duct 270 mixes with air 245 from the fan filter unit 262 to be vented back into the isolation room 225. Thus, the elongate isolation chamber 200 according to the present embodiment provides for a much larger internal chamber 220. Such an elongate isolation chamber is necessary to fit standard hospitals beds to move patients and the clinical care team within the isolation room since all other entrances into the OT are sealed.

It will be noted that when the elongate isolation chamber 200 is used together with an operating theatre, the air control system according to the operating theatre may operate separately to that of the isolation chamber 200. For instance, as an operating theatre generally operates in a positive pressure environment, venting of the air supplied to the operating theatre under normal circumstances will operate in parallel to that of the airflow control provided by the isolation chamber 200.

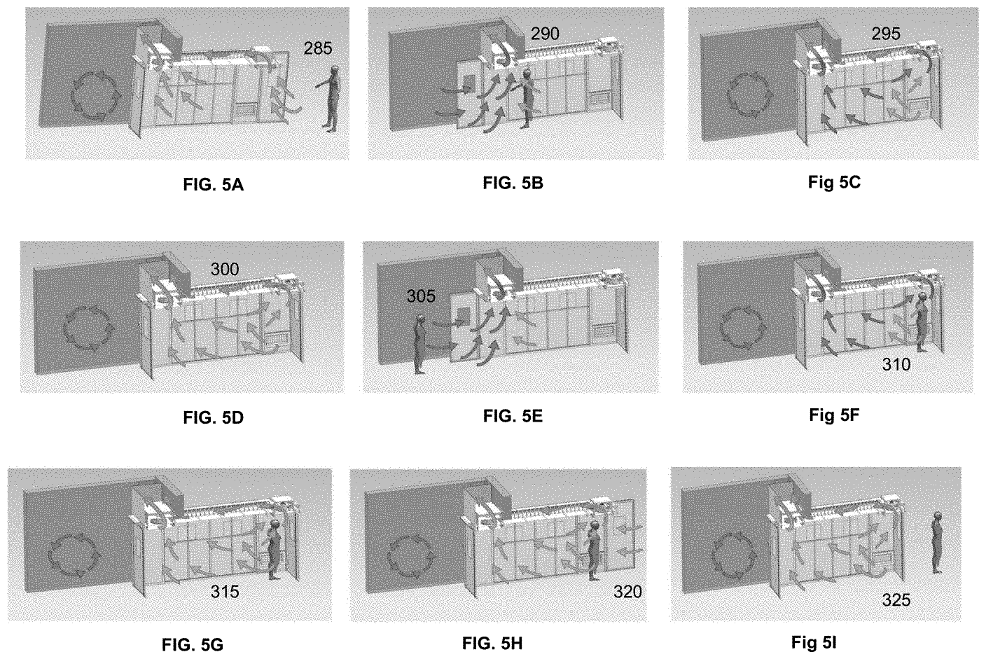

FIGS. 5A to SI show sequential steps for a person 285 entering the isolation room through the isolation chamber. Here the external door is opened, allowing air from the external environment into the internal chamber. With the door sealed, the internal door can be opened with airflow from the isolation room entering the internal chamber and redirected back into the isolation room through the fan filter unit.

FIG. 5C shows the air extraction process 295 with both doors sealed and both fan filter units directing air back into the isolation room. FIGS. 5E to SI then show a person 305 entering the isolation chamber with potentially contaminated air from the isolation room redirected back into the room. The doors are subsequently sealed and air extracted whilst the person is within the internal chamber 310. Once completed, the external door can open 320 and then resealed 325 allowing the person to exit the internal chamber and air within the internal chamber are evacuated through the two fan filter units to be redirected back into the isolation room.

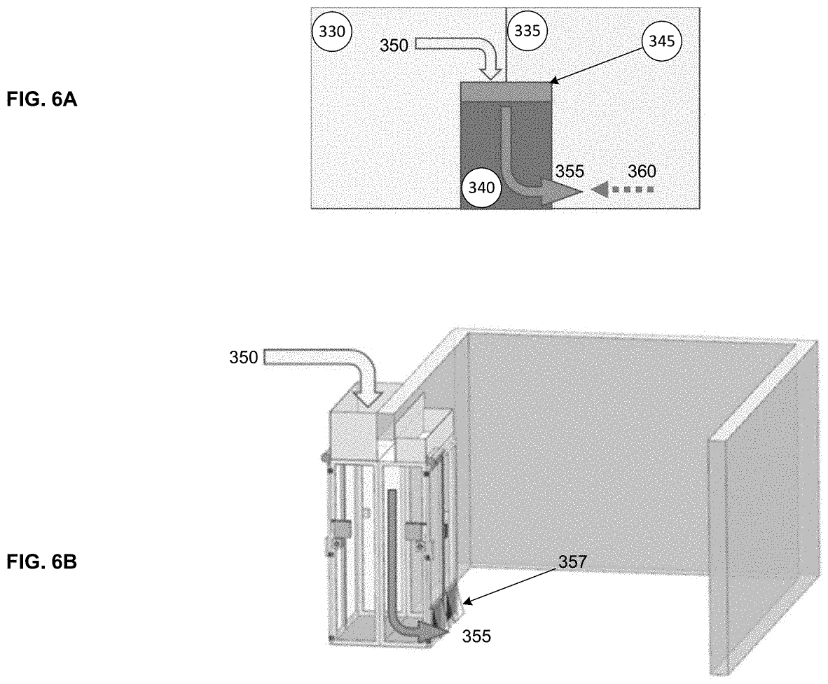

FIGS. 6 to 10 show various embodiments of the present invention under varying air flow conditions. In the following a corridor 330 and a patient room 335 are separated by an isolation chamber 340, having an FFU 345.

For FIGS. 6A and 6B, the isolation chamber has been configured as a positive pressure type (e.g. +30 Pa) to be used with positive pressured rooms 360 providing a differential pressure of, say, +15 Pa.

Air 350 is drawn from the corridor towards room (through one-way valve mechanism on room-side doors). Exhaust air flow direction is achieved due to FFU operation, creating higher isolation chamber pressure vs room's air pressure. When FFU is not in operation, the one-way valve will prevent backflow of air from the room to the isolation chamber. Allows “fresh” corridor air to be introduced 355 into the room. However, the room requires its own exhaust duct since air is constantly pumped into the room via the isolation chamber.

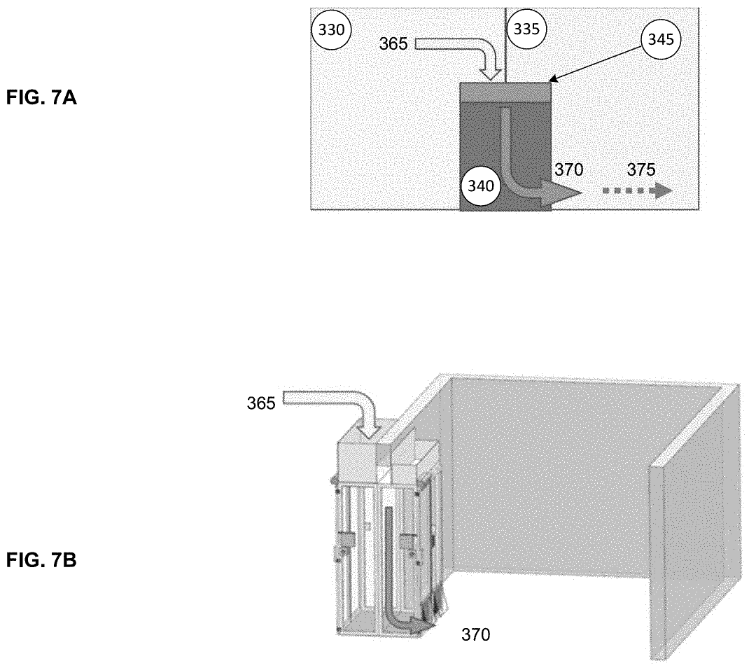

For FIGS. 7A and 7B, the isolation chamber has been configured as a positive pressure type (e.g. +30 Pa relative to corridor; +15 Pa in the isolation chamber for a −15 Pa room) for use with a negative pressure 375 room (typically for airborne infectious patients).

Air 365 is drawn from the corridor air, and vented 370 towards the room through one-way valve mechanism on room-side doors. Exhaust air flow direction is aided by FFU operation, creating higher anteroom pressure vs room's air pressure. The isolation chamber creates an interlock partition to enhance the physical isolation of room air, as well as allowing for HEPA filtered air to be introduced into the room, as opposed to normal unfiltered corridor air. This configuration allows “fresh” corridor air to be introduced into the room. However, the room requires its own exhaust duct since air is constantly pumped into the room via the anteroom.

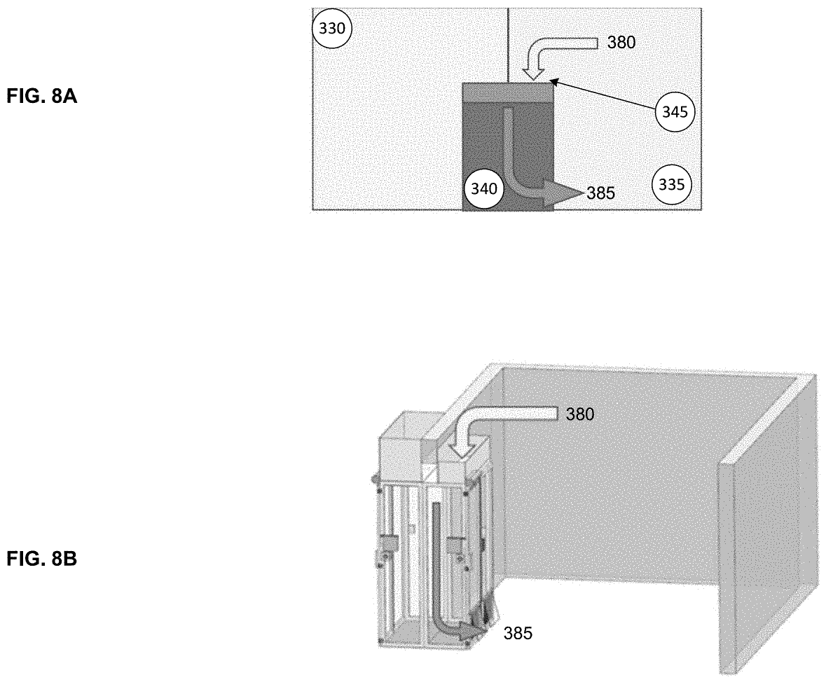

For FIGS. 8A and 8B, the isolation chamber has been configured as a positive pressure type (e.g. +15 Pa) for a neutral pressure room such as a standard room with no room air inlet or exhaust (e.g. 0 Pa). Air is drawn from room air and vented towards the room through one-way valve mechanism on room-side doors. Exhaust air flow direction is created by FFU operation, creating higher anteroom pressure vs room's air pressure.

The flow of air is recirculatory 380, 385 between the room and anteroom. Since there is no net increase in air into the room, the room does not encounter overpressurization. This eliminates the need for the room to have an exhaust duct. Anteroom creates an interlock partition to enhance the physical isolation of room air. Anteroom also allows for HEPA filtered air to be introduced into the room (as opposed to normal unfiltered corridor air).

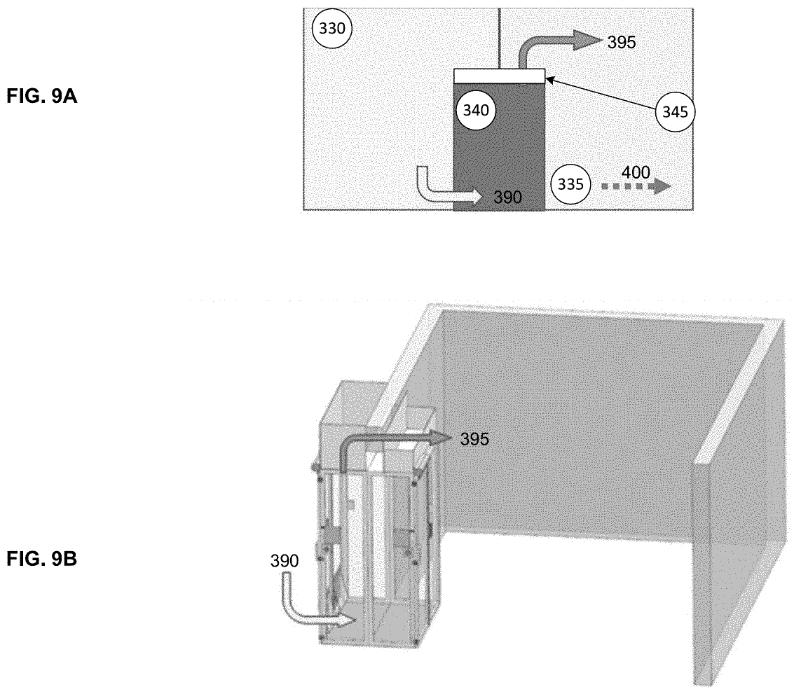

For FIGS. 9A and 9B, the isolation chamber has been configured as a negative pressure type (e.g. −15 Pa) for negative pressure rooms 400 (e.g. −15 Pa) and may be used for airborne infectious patients. Air is drawn 390 from corridor air through one-way valve mechanism on corridor-side doors, and vented 395 towards room with exhaust air flow direction is created by FFU operation.

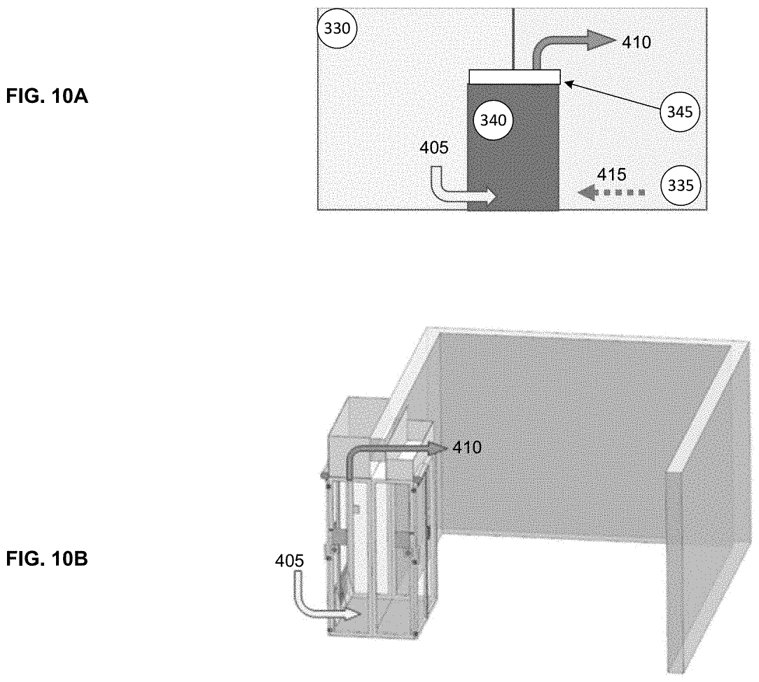

For FIGS. 10A and 10B, the isolation chamber has been configured as a negative pressure type (e.g. −15 Pa) for use with positive pressure rooms 415 (e.g. +15 Pa), such as for immunocompromised patients or operating theatre.

Air is drawn 405 from corridor air through one-way valve mechanism on corridor-side doors), and vented 410 towards the room, with exhaust air flow direction is created by FFU operation.

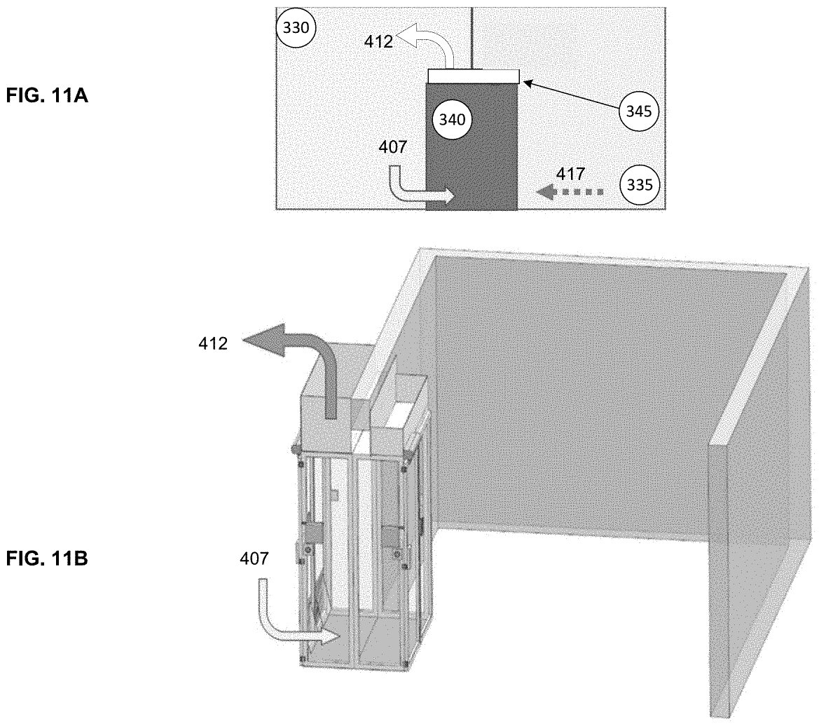

For FIGS. 11A and 11B, the isolation chamber has also been configured as a negative pressure type (e.g. −15 Pa) for use with positive pressure rooms 417 (e.g. +15 Pa), such as for immunocompromised patients or operating theatre. Air is drawn 407 from corridor air through one-way valve mechanism on corridor-side doors), and vented 412 back to the corridor, and so filtering corridor air.

FIGS. 12A and 12B show a further embodiment of the isolation chamber 420, related to the filter fan unit 430. Channeling 450 of filtered air exhaust 445 downwards over the exterior face of the room-side anteroom doors creates an air curtain effect that may assist to shed off particulates and contaminants off the users before they exit the room. The air curtain also reduces influx of room air when room-side doors are opened. It will be appreciated that the screen 425 that re-directs the air 450 may be fixed in place, so as to provide the air curtain. Alternatively, the screen 425 may be selectively adjustable to direct at other locations within the room. For instance, to ensure consistent air flow through out the room, the screen 425 may direct air to a “dead zone” where air circulation doesn't reach and thus enhance full air circulation.

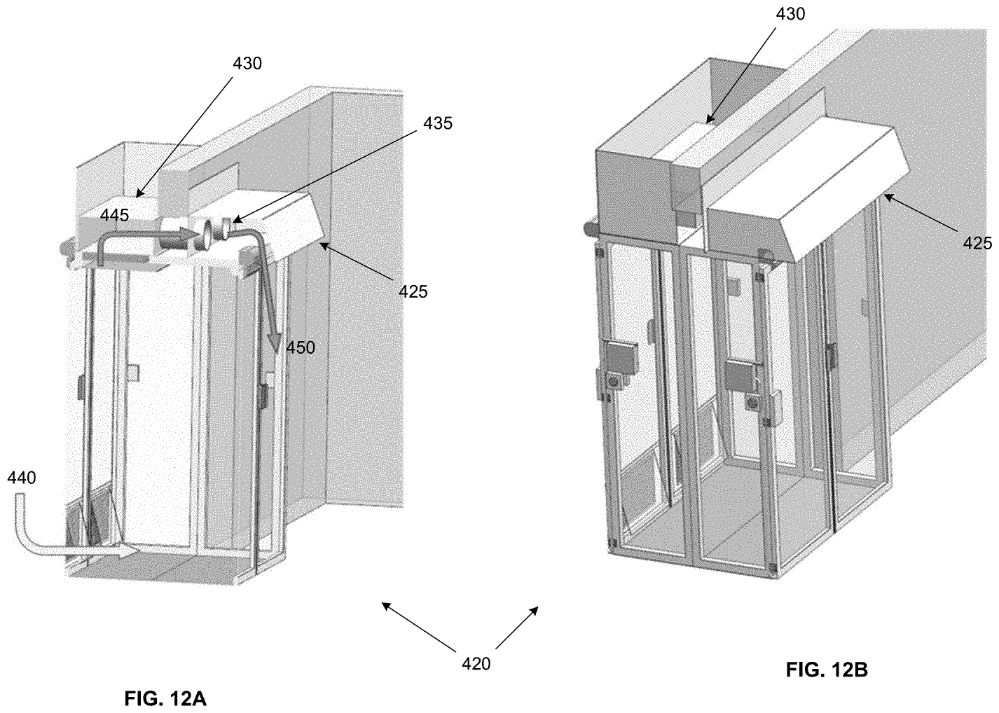

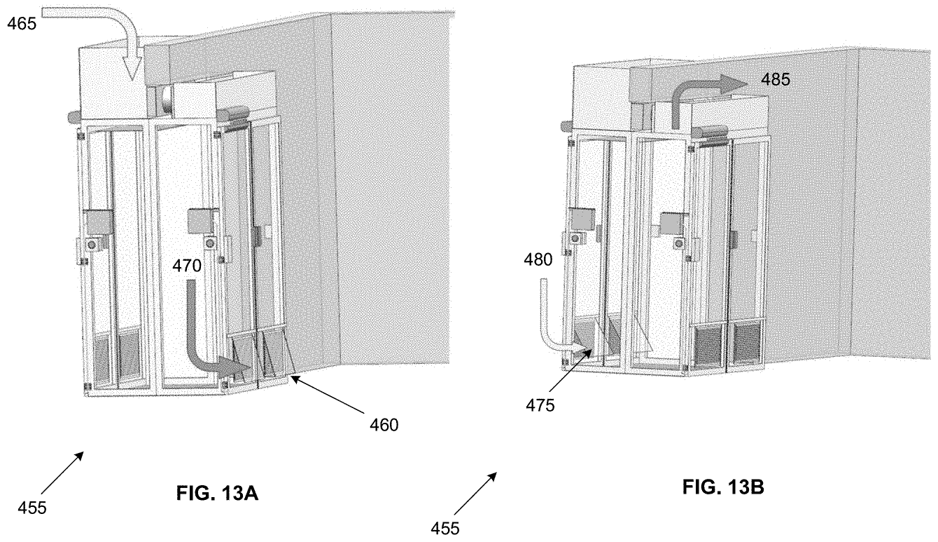

FIGS. 13A and 13B show how the isolation chamber 455 can be used in dual modes. Switching between positive and negative pressure, the isolation chamber 455 may include one-way valve flaps 460, 475 on both pairs of doors.

In positive mode, relative to the isolated room, the flaps 460 create a temporary positive pressure isolation chamber 455 prior to the opening of room-side doors. Higher isolation chamber 455 pressure and constant pumping 465 of clean air into the isolation chamber 455 reduces influx of room air when room-side doors are open.

In negative mode, relative to the corridor, the flaps 475 create a temporary negative pressure isolation chamber 455 prior to the opening of corridor-side doors. Lower isolation chamber 455 and constant exhaust of air 485 from the isolation chamber 455 reduces leak of anteroom air when corridor-side doors are open.

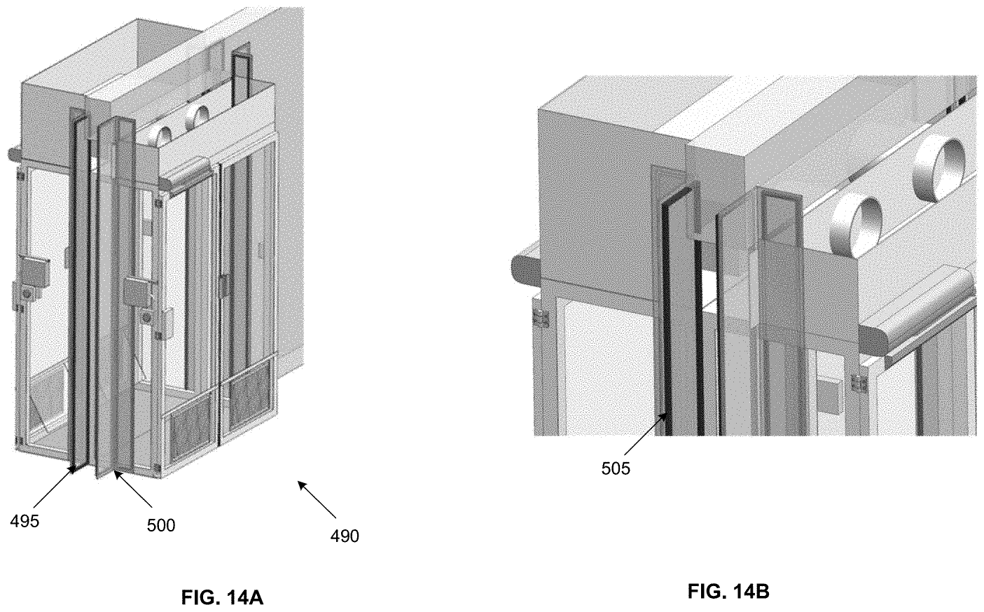

FIGS. 14A and 14B show an embodiment to selectively seal the isolation chamber 490 using at least one magnetic strip 495, 500. A better seal may be achieved using a pair of such seals 495, 500 but this will be dependent upon the circumstances. The seal includes a backing layer of a suitable flexible and impervious material such as flexible PVC, LDPE, LLDPE, silicon rubber, or other elastomer material. The backing layer includes an adhesive strip (which may be magnetically, mechanically or chemical adhesive). The strip is applied along the interface between the door opening into which the isolation chamber 490 is mounted.

In a further embodiment, striations of magnetic strips and compressible foam attached to the backing layer create a flexible and hermetically sealed surface that is readily removable and reusable. As the backing layer is a flexible material, the assembly may be attached across bends and conform to variances in gap sizes between the door and anteroom. The opposing face may include similar conjugate striations of ferrous metal that interface with the pitch of the striations. The ferrous metal strips may provide a strong fixation to the magnetic strips.

For a compressive seal, angled brackets may be are lined with compressible foam and securely fastened to the frame via bolts or other removable fasteners. The width of the angled brackets is larger than the gap between the anteroom frame and the door opening.

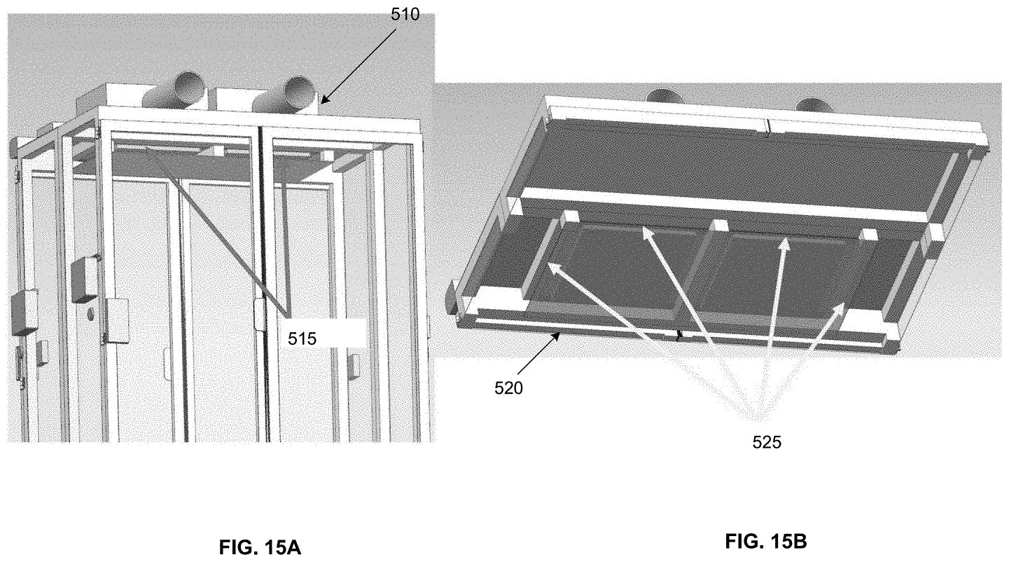

FIGS. 15 to 18 relate to various embodiments concerning decontamination.

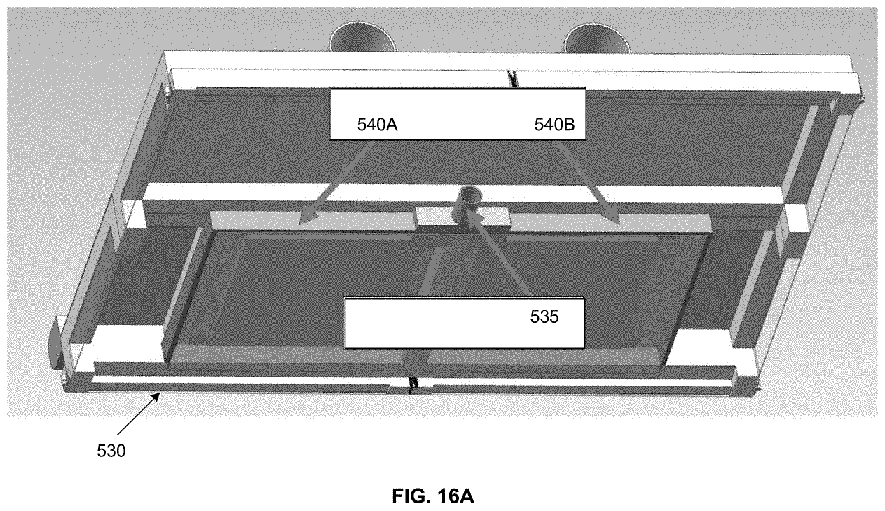

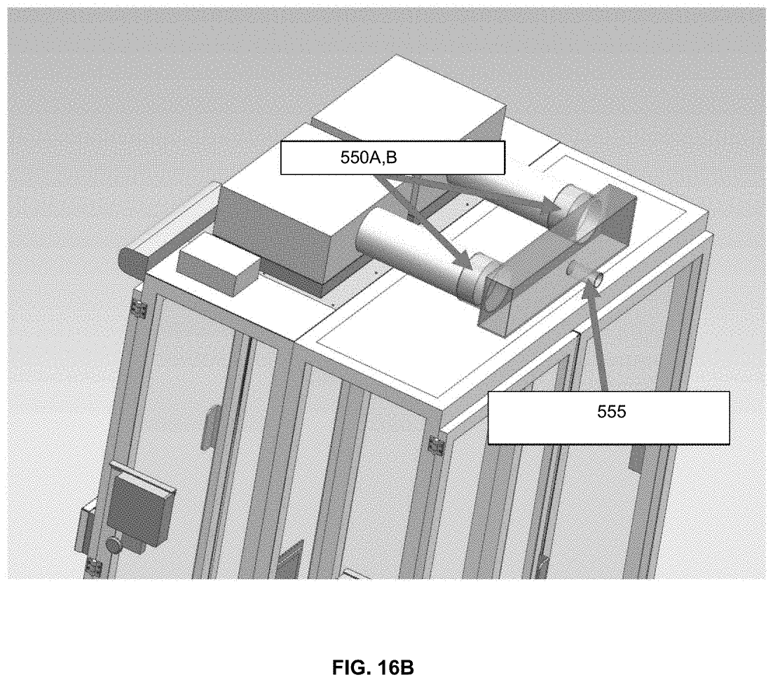

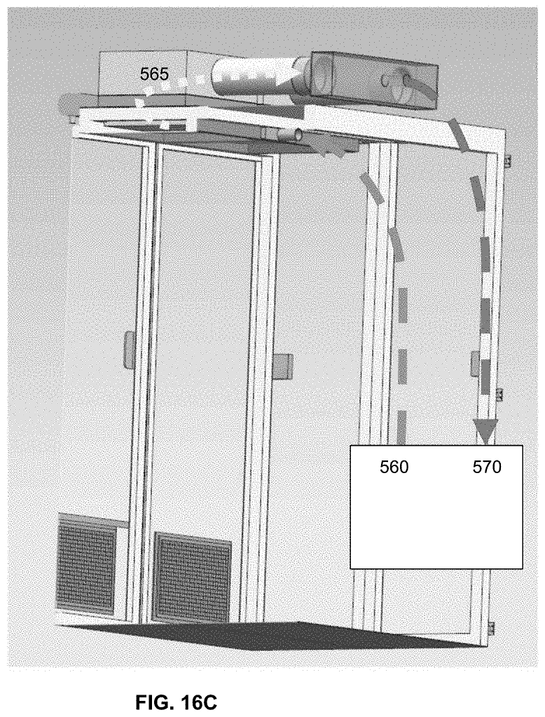

FIGS. 15A, 15B and FIGS. 16A to 16C, for instance, show an isolation chamber FFU 510 decontamination via fumigation with chemical vapor sterilant. The sterilant (such as vaporized high concentration hydrogen peroxide (VHP) 35-50%) is pumped through the FFU via the inlet ducts 525, which is collected and recirculated from the exhaust hoses of the FFU. The hydrogen peroxide vapor generator is typically a standalone machine but can optionally be installed with the isolation chamber frame for a self-contained decontamination solution

The vaporized sterilant is pumped into the inlet duct 525 of the FFU via the adapter 535 and enclosure. To return the sterilant vapor to the generator and recirculate to the inlet side, the exhaust ducts are connected to an adapter 535 and is channeled to an inlet hose towards the vapor generator.

When both the inlet and outlet hoses are connected to the vapor generator, a hermetically sealed close loop system 540A, 540B is formed between the anteroom's FFU and VHP generator.

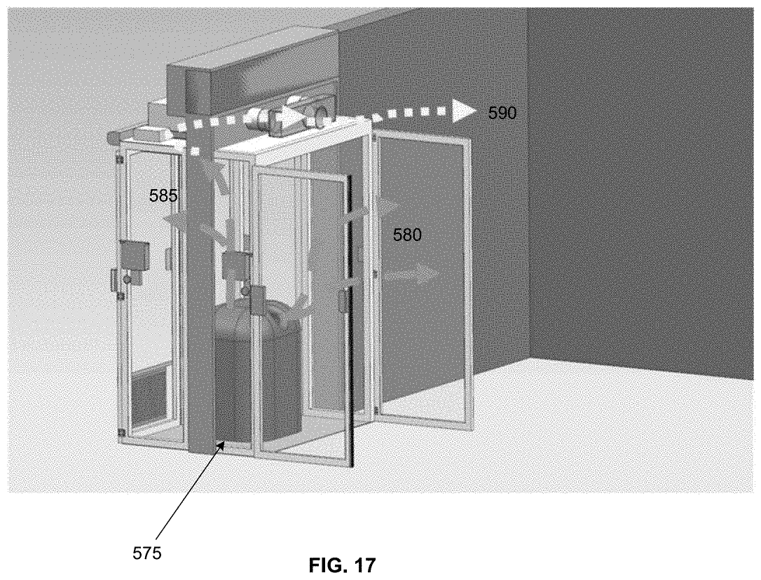

As shown in FIG. 17, room & isolation chamber decontamination via chemical vapor sterilant, may be achieved by a standalone VHP generator 575, which is placed within the isolation chamber and remotely controlled to vent 580 into the room. All gaps are taped down, and the room's inlet duct and exhaust ducts are sealed before fumigation. The corridor-side doors of the isolation chamber are closed and sealed. The room-side doors of the isolation chamber are kept open and the FFU is either programmed to run at a very low rate or the FFU is shut off and an ancillary low-speed fan is attached to the exhaust duct. The low speed flow slowly draws sterilant vapor 585 through the FFU and expels 590 it into the room. The low fan speed ensures that the sterilant completely fumigates the entire FFU's inner surfaces, achieving a high dwell time with the sterilant vapor as it passes through the FFU.

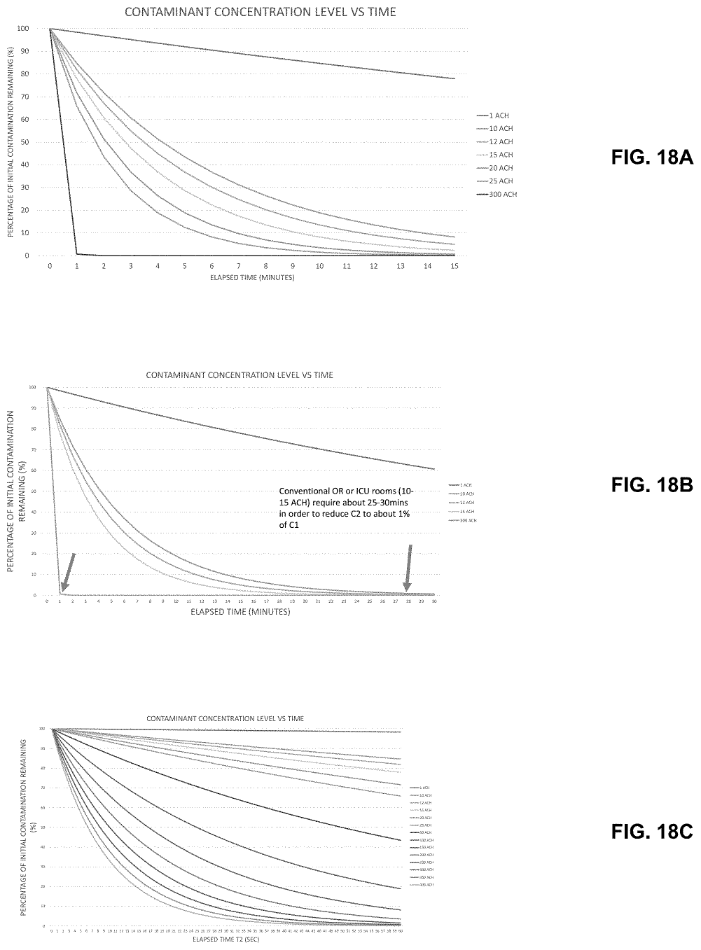

Decontamination rates may then be calculated according to the following procedure, as seen in FIGS. 18A to 18C.

$${T}_{2} - {T}_{1} = - \frac{[ ln ( {C}_{2} / {C}_{1} ) ]}{( Q / V )} * 60$$

$$Assume {T}_{1} = 0$$

$${T}_{2} = - \frac{[ ln ( {C}_{2} / {C}_{1} ) ]}{( ACH )} * 60$$

$${e}^{( - {T}_{2} * ACH / 60 )} = ( {C}_{2} / {C}_{1} )$$

Where

T₁=initial timepoint in minutes

T₂=final timepoint in minutes

C₁=initial concentration of contaminant

C₂=final concentration of contaminant

C₂/C₁=(removal efficiency/100)

Q=air flow rate in cubic metres/hour

V=room volume in cubic metres

Q/V=ACH

The rate in which a known fixed initial concentration of contaminants in a room (C₁) is diluted by a constant exchange of clean air to/from the room (ACH) is given by that formula. The higher the ACH the faster the current concentration of contaminants C₂ will drop. In terms of biological safety, it is of interest to see what dilution time T₂ is required for a certain room ACH value such that C₂ is reduced to a small fraction of C₁ (e.g. 99.9% meaning C2/C1=0.001)

For example, in one embodiment the isolation chamber may have an internal volume V of 9.43 m³ with the FFUs rated to run at 2300 m³/hr during ‘high mode’. This gives it an ACH of 244.

For the ICU embodiment of the isolation chamber, the FFU is rated to run at 1100 m³/hr at high which gives it an ACH of approximately 360. By comparison, operating theatres and isolation rooms typically have a minimum ACH of about 12-15.

Having an isolation chamber with a high ACH, it will be appreciated that the reduction in decontamination cycle time is a practical and clinical concern since patients still need to be monitored and cared for during this transit so it cannot be too long.

Additionally, if the decontamination cycle is breached, it needs to be reset.

For example, if a conventional 12 ACH anteroom is used, the decontamination cycle to achieve 99% reduction of C1 is 24 minutes long, adding to the advantages provided by the present invention.

1. An isolation chamber comprising:
a plurality of walls and ceiling defining an internal chamber;
a first and second door, the doors sealing engagement with the walls;
a fan filter unit arranged to extract air from the internal chamber;
said isolation chamber is arranged to couple to a doorway, said coupling arranged to provide an airtight seal about said doorway.

2. The isolation chamber according to claim 1, wherein the first doorway is on a first side of the doorway and the second door is on a second side of the doorway.

3. The isolation chamber according to claim 1, wherein the fan filter unit is arranged to supply air into the internal chamber from an external area corresponding to the first door corridor.

4. The isolation chamber according to claim 1, wherein any one or a combination of said walls and ceiling are articulated and arranged to expand or retract so as to fit within said doorway.

5. The isolation chamber according to claim 4, wherein the articulation includes an insertion of a portion of said chamber through said doorway, said sealing of the doorway arranged at intermediate portions of said walls and ceiling.

6. The isolation chamber according to claim 1, wherein the first and second doors are arranged to limit one door open at a time.

7. The isolation chamber according to claim 1, wherein any one or a combination of the doors, walls or ceiling may be transparent so as to provide visibility through the doorway to an observer within the chamber and/or external to the isolation chamber.

8. The isolation chamber according to claim 1, wherein the fan filter unit is arranged to be selectively retracted.

9. The isolation chamber according to claim 1, further including a control system in communication with the fan filter unit; said control system arranged to control the fan filter system so as to operate at a high flow rate for high air exchange and differential pressure to purge contaminated air.

10. The isolation chamber according to claim 9, wherein the control system is further arranged to control the fan filter system so as to operate at a low flow rate to maintain a differential pressure.

11. The isolation chamber according to claim 9, wherein the control system is arranged to detect entry/exit of personnel to the isolation chamber and to commence a decontamination cycle; including locking the doors for pre-determined time periods corresponding to decontamination.

12. The isolation chamber according to claim 9, wherein the control system is arranged to selectively operate the fan filter unit to create more localised ‘sinks’ which act to pull in contaminated air before it reaches the distal end of the isolating chamber.

13. The isolation chamber according to claim 9, wherein the control system is arranged to detect if air is being directed into the patient room, that the air is filtered and then returned.

14. The isolation chamber according to claim 9, further including a vapour generator for generating a flow of vaporised hydrogen peroxide, such that the control system is arranged to commence a room decontamination process by sealing outer doors and opening inner doors and then running the FFU to pass the vaporised hydrogen peroxide through the FFU and distributing the vaporised hydrogen peroxide into the room.
